(19) United States
(12) Patent Application Publication
(10) Pub. No.: US 2024/0352216 A1
(43) Pub. Date: Oct. 24, 2024

(54) PYROLYSIS OF A POLYMERIC POLYURETHANE COMPOUND IN ORDER TO RECOVER RAW MATERIALS

(71) Applicant: Covestro Deutschland AG, Leverkusen (DE)

(72) Inventors: Stefanie Eiden, Köln (DE); Rainer Bellinghausen, Odenthal (DE); Aurel Wolf, Wülfrath (DE); Rolf Albach, Köln (DE)

(21) Appl. No.: 18/684,233

(22) PCT Filed: Aug. 18, 2022
(86) PCT No.: PCT/EP2022/073103

§ 371 Date: Feb. 16, 2024

(30) Foreign Application Priority Data
Aug. 20, 2021 (EP) 21192326.3

(51) Int. Cl. C08J 11/12; C07C 209/62

(52) U.S. Cl. CPC C08J 11/12; C07C 209/62; C08J 2375/04

(57) ABSTRACT

The invention relates to a method according to claim 1 for the commercial implementation of a pyrolysis, and to pyrolysis devices for the pyrolysis of pyrolysis stock used in this method. The pyrolysis stock contains a polymeric compound having at least one structural polyurethane unit of the formula (I), wherein Q is a hydrocarbon group with 2 to 8 carbon atoms, preferably 3 to 8 carbon atoms, and n represents a number from 2 to 4, most preferably 2, and -* represents a covalent bond to the polymer backbone. The method and the pyrolysis device according to the invention allow, even with higher compounds of pyrolysis stock an amount of pyrolysis product to be obtained that contains cleavage products which can be reused for the synthesis of polyurethane-containing material.

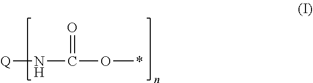

The invention relates to a pyrolysis process for the thermal utilization of polymeric polyurethane compounds, to a corresponding use of a pyrolysis device, and to the product of the pyrolysis, which comprises raw materials of a kind that can be reused for polyurethane production.

Polyurethane-containing materials find use as cushioning or as insulation materials in the refrigeration and construction sectors. Polyurethane-containing foam materials in particular are used for example as cushioning elements, mattresses or insulation material. Flexible polyurethane foam is suitable as upholstery material.

At the end of the service life of products containing polyurethane material, they are replaced with new products and normally scrapped. The total amount of plastic waste that results increases every year. About 60% of the total amount is disposed of through incineration and 40% in landfill. When incinerated, CO₂ is emitted into the air, which contributes to global warming. Its low density means that plastic waste in landfills occupies a large volume and can from there also contribute to general pollution in rivers and seas. For this reason it is important to develop an efficient recycling method with which the waste problem can be solved and at the same time allows fossil resources to be conserved. Since the abovementioned flexible polyurethane foams in particular account for only a small part—approximately 2%—of the polymer market, the recycling of polyurethanes was not an initial focus of developments. However, the continuing growth of the plastics market means it is now important to develop recycling technologies for all polymer classes in order both to reduce CO₂ emissions and to conserve fossil energy sources.

The processes for recycling plastic waste can be roughly divided into three categories:
(1) mechanical recycling: Here, the plastic waste is melted down again as is and can then be reused. This is not possible for polyurethane elastomers.
(2) chemical and thermochemical recycling: Here, the plastic waste is depolymerized into monomers or broken down into smaller molecules in order to obtain useful chemical raw materials, and
(3) thermal recycling, in which the plastic waste is converted into offgas and heat energy.

The chemical raw materials obtained from thermochemical recycling can be used to synthesize new synthetic resins or other chemical products.

Thermochemical recycling is referred to as pyrolysis. In most cases pyrolysis is employed for packaging waste, affording a pyrolysis oil that is used as a kind of recycled naphtha in known refinery processes with crackers in the form of a drop-in solution. The pyrolysis of polyurethanes is something about which little is known.

The use of catalysts or additives in the pyrolysis process can lower operating temperatures, shorten reaction times, increase degradation efficiency, and restrict product distribution, making the process more efficient.

Garrido et al. (Chemosphere 168 (2017), 667-675) describe the decomposition of flexible polyurethane foam in a temperature window of 300 to 850° C. without subsequent condensation and isolation of the pyrolysis product: the formation of isocyanate species was detected by instrumental analysis.

Hileman et al. (J. of Polymer Science, Polymer Chemistry Edition 13 (1975), 571-584) carried out the pyrolysis of commercially available flexible polyurethane foam at temperatures of 300° C., 500° C., 750° C., and 1000°° C. without subsequent condensation and isolation of the pyrolysis product. The isocyanate component (in this case TDI), which can hydrolyze to the corresponding amine, was observed to form in greatest amount at a temperature of 500° C. According to Hileman et al., TDI and the corresponding amines are thermally stable at temperatures below 500° C. but undergo increasing decomposition above a temperature of 750° C.

Ravey et al. (J. of Applied Polymer Science, 63 (1997), 47-74) describe the thermal decomposition of flexible polyurethane foam on a microscale at a temperature of 320 to 360° C. without subsequent condensation and isolation of the pyrolysis product: the formation of aromatic isocyanate (in this case TDI) or aromatic amine (in this case DAT) was detected in the gas chromatograph.

Kumagaia et al. (Journal of Analytical and Applied Pyrolysis 126 (2017) 337-345) describe a pyrolysis of flexible and rigid foams carried out at approx. 800° C. in which isocyanates, diamines, and a great many other fragments from the polyol are obtained.

DE 2410505 C2 discloses a process for the thermal production of isocyanates from urethanes at 400 to 530° C. and reduced pressure. There was no mention of experiments with polyurethanes at normal pressure.

DE 2362915 proposes the hydrolysis of flexible polyurethane foams to diamines in a fluid bed.

None of the pyrolysis processes described in the prior art are suitable for industrial or commercial use, since they are mostly intended for use as pyrolyses on a microgram scale for analytical purposes. The pyrolyses presented in the prior art are therefore in each case carried out in pyrolysis devices that are not suitable for industrial or commercial use.

Particularly for the pyrolysis of larger amounts of polyurethane-containing materials, there is a need for novel, more selective processes with suitable reactors, with which, through appropriate process control, the formation of undesired by-products can be reduced. Polyurethane-containing materials should selectively afford pyrolysis products having a high content of cleavage products that can be reused for polyurethane synthesis, especially aromatic amine compounds such as aniline and/or toluidine and/or TDA.

The object was therefore to provide, for the commercial execution of a pyrolysis, a process and pyrolysis devices employable in said process for the pyrolysis of a pyrolysis feedstock at least including polyurethane-containing material, the use of which affords, even with relatively large amounts of pyrolysis feedstock, an amount of a pyrolysis product that comprises cleavage products that can be reused for the synthesis of polyurethane-containing material, preferably amino-group-containing cleavage products in an amount of more than 10% by weight based on the total weight of polyurethane-containing material used. A recovery of at least 40% by weight of amino-group-containing cleavage products based on the total weight of the organic isocyanate used for the production of the polyurethane-containing polymeric compound is here.

The present application therefore provides a pyrolysis process, comprising at least the following steps:
(a) introducing a pyrolysis feedstock comprising at least one polymeric compound having at least one polyurethane structural unit of the formula (I),

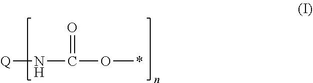

where
Q is a hydrocarbon radical having 2 to 8 carbon atoms, preferably 3 to 8 carbon atoms, and
n is a number from 2 to 4, more preferably 2,
and -* denotes a covalent bond to the polymer backbone, into a reactor:
(b) decomposing at least the polymeric compound of the pyrolysis feedstock introduced in step (a) in the reactor at a temperature of 200° C. to 350° C. to obtain a gas-phase product as pyrolysate and a non-gas-phase pyrolysis residue, wherein
   (i) during said decomposition, the amount of oxygen gas in the reactor is not more than 2.0% by volume based on the total volume of the gases present in the reactor, and
   (ii) during said decomposition, the pyrolysate is discharged from the reactor, and
   (iii) said pyrolysis residue is discharged from the reactor:
(c) cooling the discharged pyrolysate to a temperature of less than 200° C. to obtain pyrolysis product selected from pyrolysate condensate, pyrolysate sublimate or a mixture thereof:
(d) optionally working up the pyrolysis product.

The term “pyrolysis feedstock” refers to the totality of the substances introduced into the reactor for the pyrolysis that therein undergo thermal treatment in the absence of oxygen gas or in the presence of a reduced amount of oxygen gas. The pyrolysis feedstock is preferably in solid form prior to its introduction into the reactor.

“Pyrolysate” is understood as meaning the totality of the products formed by pyrolysis that are in the gas phase in the reactor under the conditions of step (b) (more particularly in the form of a gas and/or as an aerosol).

“Pyrolysis residue” is understood as meaning the totality of the substances formed by pyrolysis and other residues of the pyrolysis feedstock that are not in the gas phase in the reactor under the conditions of step (b). Preferred embodiments of the process are those in which the pyrolysis residue in the reactor is solid or liquid.

“Pyrolysis product” is understood as meaning the totality of the products from the pyrolysate that in step (c) accumulate by condensation and/or resublimation when the pyrolysate cools. Pyrolysis product that is liquid is also referred to as pyrolysis oil.

Unless explicitly otherwise defined in this connection, a substance (for example material, pyrolysis feedstock, pyrolysate, pyrolysis product, pyrolysis residue) is “liquid” if it is in the liquid state at 20° C. and 1013 mbar. Unless explicitly otherwise defined in this connection, a substance (for example material, pyrolysis feedstock, pyrolysate, pyrolysis product, pyrolysis residue) is “solid” if it is in the solid state at 20° C. and 1013 mbar. Unless explicitly otherwise defined in this connection, a substance (for example material, pyrolysis feedstock, pyrolysate, pyrolysis residue) is “gaseous” if it is present as a gas at 20° C. and 1013 mbar.

A substance is “organic” if its chemical structure includes at least one covalent carbon-hydrogen bond.

In this application, the average molar masses specified for polymers or for polymer ingredients are unless explicitly otherwise stated always weight-average molar masses Mw, which can in principle be determined by gel-permeation chromatography using an RI detector, it being expedient to perform the measurement against an external standard.

A “reactor” is a volume in which a chemical transformation, for example a thermal decomposition of material from the pyrolysis feedstock, takes place. For a thermal decomposition this can for example be the volume of a heated vessel in which the pyrolysis feedstock is contained.

It is in accordance with the invention advantageous when the pyrolysis feedstock is in accordance with the process of the invention introduced into a reactor characterized in that it is selected from a continuous stirred-tank reactor (CSTR), fixed-bed reactor, fluid-bed reactor, screw reactor, screw-conveyor reactor, entrained-flow reactor, entrainment-flow reactor, rotary-tube reactor, fluid-bed reactor, and drum reactor. More particularly, suitable reactors are preferably ones in which the pyrolysis feedstock can be introduced continuously and are selected in particular from a rotary-tube reactor, continuous stirred-tank reactor (CSTR), fixed-bed reactor (especially with continuous bed exchange (shaft reactor) with internal heat exchangers, preferably with internal heat exchanger tubes), screw reactor, screw-conveyor reactor, entrained-flow reactor, rotary-tube reactor or fluidized-bed reactor. In one embodiment of the process a very particularly preferred reactor is selected from a screw reactor, a rotary oven or fluidized bed. Further reactors preferred for the process of the invention and embodiments thereof are described in the embodiments of the pyrolysis device of the invention and in an embodiment of the process with the use of a catalyst (vide infra).

According to the invention, the pyrolysis feedstock introduced into the reactor includes at least one polymeric compound having at least one polyurethane structural unit of the formula (I),

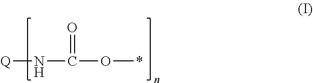

where
Q is a hydrocarbon radical having 2 to 8 carbon atoms, preferably 3 to 8 carbon atoms, and
n is a number from 2 to 4, more preferably 2,
and -* denotes a covalent bond to the polymer backbone.

Q is preferably derived from aliphatic hydrocarbon units, cycloaliphatic hydrocarbon units, araliphatic hydrocarbon units, aromatic hydrocarbon units or heterocyclic hydrocarbon units.

The process of the invention is particularly well suited to materials that comprise a polymeric compound of the aforementioned kind in which Q according to formula (I) includes an aromatic radical.

A very particularly preferred embodiment of the process of the invention is characterized in that the polymeric compound contains at least one structural unit of the formula (Ia)

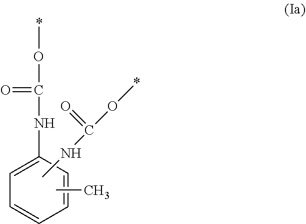

where
-* is a covalent bond to the polymer backbone.

The process as claimed in any of the preceding claims, characterized in that the polymeric compound is obtained by

The polymeric compound present in said material can preferably be obtained by reaction at least of
i1) at least one organic isocyanate compound containing at least two to four, especially two, isocyanate groups attached to a hydrocarbon unit having 2 to 8 carbon atoms, preferably having 3 to 8 carbon atoms; with
i2) at least one organic compound having at least two hydroxy groups.

It is in a further embodiment preferable when the at least one organic isocyanate compound contains, as said hydrocarbon unit, a unit that has the number of carbon atoms stated in i1) and is derived from aliphatic hydrocarbon units, cycloaliphatic hydrocarbon units, araliphatic hydrocarbon units, aromatic hydrocarbon units or heterocyclic hydrocarbon units.

Particularly preferably, at least one compound corresponding to formula (II) is selected as said organic isocyanate compound

Q(NCO)ₙ (II)

where n is a number from 2 to 4, preferably from 2 to 3, and Q is a radical selected from an aliphatic hydrocarbon radical having 2 to 8 carbon atoms, preferably having 3 to 8 carbon atoms, a cycloaliphatic hydrocarbon radical having 3 to 8 carbon atoms, an aromatic hydrocarbon radical having 6 to 8 carbon atoms, preferably having 10 to 30 carbon atoms or an araliphatic hydrocarbon radical having 6 to 8 carbon atoms.

When said polymeric compound contains at least one structural unit of the above formula (Ia) embodiment, a further preferred embodiment of the process has been found to be when at least one diisocyanate of the formula (III) is selected as the at least one organic polyisocyanate compound in step i1),

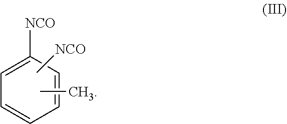

A preferred embodiment of the above step i2) is when at least one organic compound having at least two hydroxy groups is selected from polyester polyol, polyether polyol, polycarbonate polyol, polyetherester polyol, polyacrylate polyol, polyester polyacrylate polyol or mixtures thereof, more preferably selected from the group comprising polyether polyols and/or polyester polyols.

The OH value of the employed organic compound having at least two hydroxy groups or employed organic compounds having at least two hydroxy groups, in each case according to DIN 53240-1 (June 2013), is preferably from 15 to 4000 mg KOH/g. When more than just one organic compound having at least two hydroxy groups is used, the mixture of said compounds may preferably have a hydroxyl value of between 20 to 200 mg KOH/g, especially 25 to 100 mg KOH/g. In the case of a single added organic compound having at least two hydroxy groups, the OH value (or hydroxyl value) indicates the OH value of said compound. Reported OH values for mixtures relate to the number-average OH value of the mixture, calculated from the OH values of the individual components in their respective molar proportions. The OH value indicates the amount of potassium hydroxide in milligrams that is equivalent to the amount of acetic acid bound by one gram of substance on undergoing acetylation. It is determined in the context of the present descriptions according to the DIN 53240-1 standard (June 2013).

The organic compounds having at least two hydroxy groups preferably have a number-average molecular weight of ≥100 g/mol to ≤15 000 g/mol, especially ≥2000 g/mol to ≤12 000 g/mol, and more preferably ≥3500 g/mol to ≤6500 g/mol.

The number-average molar mass Mn (or molecular weight) is determined in the context of these descriptions by gel-permeation chromatography according to DIN 55672-1 of August 2007, unless explicitly stated otherwise elsewhere.

The organic compounds having at least two hydroxyl groups have a functionality of 1 to 8, where “functionality” in the context of the present invention refers to the theoretical average functionality (number of isocyanate-reactive or polyol-reactive functions in the molecule) calculated from the known input materials and proportions thereof.

Employable polyetherester polyols are compounds containing ether groups, ester groups, and OH groups. For the production of the polyetherester polyols, preference is given to using organic dicarboxylic acids having up to 12 carbon atoms, preferably aliphatic dicarboxylic acids having 4 to 6 carbon atoms or aromatic dicarboxylic acids, used individually or in a mixture. Examples include suberic acid, azelaic acid, decanedicarboxylic acid, malonic acid, phthalic acid, pimelic acid, and sebacic acid and especially glutaric acid, fumaric acid, succinic acid, adipic acid, phthalic acid, terephthalic acid, and isophthalic acid. In addition to organic dicarboxylic acids, derivatives of these acids can also be used, for example their anhydrides and also the esters and monoesters thereof with low-molecular-weight monofunctional alcohols having 1 to 4 carbon atoms. The use of proportions of the abovementioned biobased starting materials, especially of fatty acids/fatty acid derivatives (oleic acid, soy bean oil, etc.), is likewise possible and can have advantages, for example in respect of storage stability of the polyol formulation, dimensional stability, fire behavior, and compressive strength of the foams.

Polyether polyols obtained by alkoxylation of starter molecules such as polyhydric alcohols are a further component used for producing polyether ester polyols. The starter molecules are at least difunctional, but may optionally also contain proportions of higher-functionality, especially trifunctional, starter molecules.

Examples of starter molecules include ethylene glycol, propylene glycol, butane-1,3-diol, butane-1,4-diol, pentene-1,5-diol, pentane-1,5-diol, hexane-1,6-diol, heptane-1,7-diol, octane-1,8-diol, decane-1,10-diol, 2-methylpropane-1,3-diol, neopentyl glycol, 2,2-dimethylpropane-1,3-diol, 3-methylpentane-1,5-diol, 2-butyl-2-ethylpropane-1,3-diol, 2-butene-1,4-diol and 2-butyne-1,4-diol, diethylene glycol, dipropylene glycol, glycerol, trimethylolpropane, and di-and trifunctional polyether polyols. The polyether polyols preferably have an OH functionality of 2 to 4 and a molecular weight Mn in the range from 62 to 4500 g/mol and especially a molecular weight Mn in the range from 62 to 3000 g/mol. Starter molecules having functionalities other than OH can also be used, alone or in a mixture.

Polyether ester polyols may also be produced by the alkoxylation, especially by ethoxylation and/or propoxylation, of reaction products obtained by the reaction of organic dicarboxylic acids and derivatives thereof as well as components having Zerewitinoff-active hydrogens, especially diols and polyols. Derivatives of such acids that may be used include, for example, their anhydrides.

The polyester polyols may for example be polycondensates of polyhydric alcohols, preferably diols, having 2 to 12 carbon atoms, preferably having 2 to 6 carbon atoms, and polycarboxylic acids, for example di-, tri-or even tetracarboxylic acids or hydroxycarboxylic acids or lactones, with preference given to the use of aromatic dicarboxylic acids or mixtures of aromatic and aliphatic dicarboxylic acids. Instead of the free polycarboxylic acids it is also possible to use the corresponding polycarboxylic anhydrides or corresponding polycarboxylic esters of lower alcohols for production of the polyesters.

Useful carboxylic acids include in particular: succinic acid, glutaric acid, adipic acid, suberic acid, azelaic acid, sebacic acid, decanedioic acid, tetrahydrophthalic acid, hexahy drophthalic acid, cyclohexanedicarboxylic acid, tetrachlorophthalic acid, itaconic acid, malonic acid, 2-methylsuccinic acid, 3,3-diethylglutaric acid, 2,2-dimethylsuccinic acid, dodecanedioic acid, endomethylenetetrahydrophthalic acid, dimer fatty acid, trimer fatty acid, citric acid, trimellitic acid, benzoic acid, maleic acid, fumaric acid, phthalic acid, isophthalic acid, and terephthalic acid. It is likewise possible to use derivatives of these carboxylic acids, for example dimethyl terephthalate. The carboxylic acids may be used either individually or in a mixture. Carboxylic acids used with preference are adipic acid, sebacic acid and/or succinic acid, more preferably adipic acid and/or succinic acid.

Hydroxy carboxylic acids that may be co-used as co-reactants in the production of a polyester polyol having terminal hydroxyl groups include for example lactic acid, malic acid, hydroxycaproic acid, hydroxy butyric acid, hydroxy decanoic acid, hydroxystearic acid, and the like. Suitable lactones include caprolactone, butyrolactone, and homologs.

Also especially useful for production of the polyester polyols are biobased starting materials and/or derivatives thereof, for example castor oil, polyhydroxy fatty acids, ricinoleic acid, hydroxyl-modified oils, grapeseed oil, black cumin oil, pumpkin seed oil, borage seed oil, soy bean oil, wheat germ oil, rapeseed oil, sunflower seed oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, fatty acids, hydroxyl-modified and epoxidized fatty acids and fatty acid esters, for example based on myristoleic acid, palmitoleic acid, oleic acid, vaccenic acid, petroselic acid, gadoleic acid, erucic acid, nervonic acid, linoleic acid, alpha-and gamma-linolenic acid, stearidonic acid, arachidonic acid, timnodonic acid, clupanodonic acid, and cervonic acid. Particular preference is given to esters of ricinoleic acid with polyfunctional alcohols, for example glycerol. Preference is also given to the use of mixtures of such biobased acids with other carboxylic acids, for example phthalic acids.

Examples of suitable diols are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate. Preference is given to using ethylene glycol, diethylene glycol, butane-1,4-diol, pentane-1,5-diol, hexane-1,6-diol or mixtures of at least two of said diols, especially mixtures of butane-1,4-diol, pentane-1,5-diol, and hexane-1,6-diol.

It is additionally also possible to use polyols such as trimethylolpropane, glycerol, erythritol, pentaerythritol, trimethylolbenzene or tris (hydroxyethyl) isocyanurate, with preference given to glycerol and trimethylolpropane.

The polyether polyols that may be used as an organic compound having at least two hydroxy groups are obtained by methods of preparation known to those skilled in the art, for example by anionic polymerization of one or more alkylene oxides having 2 to 4 carbon atoms with alkali metal hydroxides, such as sodium or potassium hydroxide, alkali metal alkoxides, such as sodium methoxide, sodium or potassium ethoxide or potassium isopropoxide, or aminic alkoxylation catalysts, such as dimethylethanolamine (DMEA), imidazole and/or imidazole derivatives, or DMC catalysts using at least one starter molecule having 2 to 8, preferably 2 to 6, attached reactive hydrogen atoms.

Examples of suitable alkylene oxides are tetrahydrofuran, 1,3-propylene oxide, 1,2- and 2,3-butylene oxide, styrene oxide and preferably ethylene oxide and 1,2-propylene oxide. The alkylene oxides may be used individually, alternately one after the other, or as mixtures. Preferred alkylene oxides are propylene oxide and ethylene oxide, with particular preference given to copolymers of propylene oxide with ethylene oxide. The alkylene oxides may be reacted in combination with CO₂.

Examples of useful starter molecules include: water, organic dicarboxylic acids, such as succinic acid, adipic acid, phthalic acid, and terephthalic acid, aliphatic and aromatic, optionally N-mono-, N,N-and N,N′-dialkyl-substituted diamines having 1 to 4 carbon atoms in the alkyl radical, such as optionally mono-and dialkyl-substituted ethylenediamine, diethylenetriamine, triethylenetetramine, 1,3-propylenediamine, 1,3- and 1,4-butylenediamine, 1,2-, 1,3-, 1,4-, 1,5-, and 1,6-hexamethylenediamine, phenylenediamines, 2,3-, 2,4-, and 2,6-tolylenediamine, and 2,2′-, 2,4′-, and 4,4′-diaminodiphenylmethane. Preference is given to using dihydric or polyhydric alcohols, such as ethanediol, propane-1,2- and -1,3-diol, diethylene glycol, dipropylene glycol, butane-1,4-diol, hexane-1,6-diol, paraformaldehyde, triethanolamine, bisphenols, glycerol, trimethylolpropane, pentaerythritol, sorbitol, and sucrose.

Employable polycarbonate polyols include hydroxyl-containing polycarbonates, for example polycarbonate diols. These are formed in the reaction of carbonic acid derivatives, such as diphenyl carbonate, dimethyl carbonate or phosgene, with polyols, preferably diols.

Examples of such diols are ethylene glycol, propane-1,2- and -1,3-diol, butane-1,3- and -1,4-diol, hexane-1,6-diol, octane-1,8-diol, neopentyl glycol, 1,4-bishydroxy methylcyclohexane, 2-methylpropane-1,3-diol, 2,2,4-trimethylpentane-1,3-diol, dipropylene glycol, polypropylene glycols, dibutylene glycol, polybutylene glycols, bisphenols, and lactone-modified diols of the abovementioned type.

Also employable instead of or in addition to pure polycarbonate diols are polyether-polycarbonate diols obtainable for example by copolymerization of alkylene oxides, for example propylene oxide, with CO₂.

As compounds having isocyanate-reactive hydrogen atoms, it is also possible to use polymer polyols, PHD polyols, and PIPA polyols as an organic compound having at least two hydroxy groups. Polymer polyols are polyols containing proportions of solid polymers produced by free-radical polymerization of suitable monomers such as styrene or acrylonitrile in a base polyol. PHD (polyhydrazodicarbonamide) polyols are produced for example by in-situ polymerization of an isocyanate or an isocyanate mixture with a diamine and/or hydrazine (or hydrazine hydrate) in a polyol, preferably a polyether polyol. The PHD dispersion is preferably produced by reaction of an isocyanate mixture of 75% to 85% by weight of 2,4-tolylene diisocyanate (2,4-TDI) and 15% to 25% by weight of 2,6-tolylene diisocyanate (2,6-TDI) with a diamine and/or hydrazine hydrate in a polyether polyol produced by alkoxylation of a trifunctional starter (for example glycerol and/or trimethylolpropane). PIPA polyols are polyether polyols modified with alkanolamines by polyisocyanate polyaddition, wherein the polyether polyol preferably has a functionality of from 2.5 to 4.0 and a hydroxyl value of from 3 mg KOH/g to 112 mg KOH/g (molecular weight from 500 to 18000 g/mol).

It is also possible to use isocyanate-reactive substances having a cell opening effect, for example copolymers of ethylene oxide and propylene oxide having an excess of ethylene oxide or aromatic diamines such as diethyltoluenediamine.

For the production of polyurethane foams in the cold-cure foam process, polyethers having at least two hydroxyl groups and an OH value of 20 to 50 mg KOH/g are in a further embodiment employed as an organic compound having at least two hydroxy groups, wherein at least 80 mol % of the OH groups are primary OH groups (determination by ¹H NMR (for example Bruker DPX 400, deuterochloroform)). It is particularly preferable when the OH value is 25 to 40 mg KOH/g, very particularly preferably 25 to 35 mg KOH/g. In addition to the structural unit of the formula (I) or (Ia) defined above, the polymeric compound of the material may optionally additionally contain an isocyanurate structural unit of the following formula

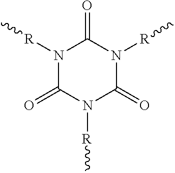

where R is a divalent hydrocarbon radical, especially a divalent aromatic hydrocarbon radical. For this embodiment it has been found to be preferable when the proportion of this isocyanurate structural unit is not more than 1% by weight of the total weight of the material.

The material comprising said polymeric compound is preferably a foam, more preferably a polyurethane foam. When the material is present in the form of a polyurethane foam, it is in turn preferably a flexible polyurethane foam or a rigid polyurethane foam. A flexible polyurethane foam is a very particularly preferred embodiment of the polymeric compound introduced in the pyrolysis feedstock in step a) of the process.

When said polymeric compound is present in the form of a flexible polyurethane foam, it has been found to be advantageous when the flexible polyurethane foam has in accordance with DIN 7726:1982-05, at a compressive load for 10% compression, a compressive stress of ≤15 kPa, measured according to DIN 53421:1984-06.

It has been found to be advantageous when said polymeric compound in the pyrolysis feedstock is introduced into the reactor preferably in the form of solid particles (especially in the form of a granular mixture). A corresponding granular mixture is formed from a multitude of loose, solid particles, which in turn comprise what are known as grains. A grain is a term for the particulate constituents of powders (grains are the loose, solid particles), dusts (grains are the loose, solid particles), granules (loose, solid particles are agglomerates of several grains), and other granular mixtures. The flowability of a granular mixture relates to its ability to flow freely under its own weight from a pour test funnel having an outlet 16.5 mm in diameter.

Said solid particles, more particularly the loose, solid particles of the granular mixture, preferably have a median diameter X_{50.3} (volume average) of from 0.01 mm to 5 cm, preferably from 0.1 mm to 5 cm. The median particle size diameter X_{50.3} is determined by sieving or using a Camsizer particle size analyzer from Retsch.

The metering and processibility of the pyrolysis feedstock in the pyrolysis of the process of the invention can be made easier when said pyrolysis feedstock in step (a) additionally comprises besides said polymeric compound at least one filler. Said filler preferably does not catalyze the thermal decomposition of polyurethane during the pyrolysis. It is in turn particularly preferable when the filler is at least one metal oxide not catalytically active in the pyrolysis in the thermal decomposition of polyurethane that is preferably selected from SiO₂.

Said material is preferably mixed with a filler, for example sand, which makes continuous process control of the process of the invention easier. In particular, sticking of said polymeric compound in the reactor and in the supply of the metering device to the reactor is avoided. In one embodiment, the filler and said polymeric compound are supplied to the pyrolysis feedstock as a mixture in a volume ratio of filler to said polymeric compound of at least 0.1:1 to 10:1.

The pyrolysis material may additionally comprise at least one catalyst. This catalyst influences the decomposition reaction of said polymeric compound. A suitable catalyst can lower the pyrolysis temperature, reduce the spectrum of products to the desired products, and optionally minimize carbonization. For an efficient process, inexpensive catalysts are preferred.

Catalysts may be naturally occurring materials such as inorganic salts, refractory oxides, minerals, and industrial stones into which ions can optionally be exchanged in a simple ion-exchange process, since they do not require extensive synthesis. They are readily available and therefore relatively cheap. Synthetic catalysts such as zeolites (ZSM-5, zeolite X, Y, etc.) are on the other hand an example of catalysts that, although effective, are not cheap, since they have to be specially manufactured.

In addition to their function as a catalyst, catalysts can also make the metering and processibility of the pyrolysis feedstock in the pyrolysis easier. The use of a catalyst allows the amount of filler used to be reduced. In one embodiment, the total amount of filler and catalyst in relation to the amount of said material is supplied to the pyrolysis feedstock as a mixture in a volume ratio of at least 0.1:1 to 10:1.

The pyrolysis feedstock introduced into the reactor in step (a) is at least partially broken down according to step (b), with the formation of pyrolysate and pyrolysis residue. After being introduced into the reactor, the pyrolysis feedstock is heated to a temperature in the range from 200° C. to 350° C. A particularly successful improvement of the result of the process of the invention can be achieved when, in one embodiment, the introduced pyrolysis feedstock is temperature-controlled at 200° C. to 350° C. and, on reaching this target temperature, the residence time of the correspondingly temperature-controlled pyrolysis feedstock until the time of discharge of the pyrolysis residue resulting therefrom is from 1 second to 2 hours, preferably between 2 minutes and 60 minutes, the temperature and content of oxygen gas in the reactor during this time being the values defined in step (b).

Independently thereof, good results can be achieved when the discharge of the pyrolysate from the reactor that takes place in step (b) is ensured by a gas stream passed through the reactor or by suction, and more preferably by the residence time of the pyrolysate, as the period between the time of introduction of said material introduced into the reactor in step (a) and the time of discharge of the pyrolysate, being from 0.1 seconds to 600 seconds, preferably between 0.5 seconds and 300 seconds, more preferably 0.5 seconds to 200 seconds.

If a gas stream is passed through the reactor for this purpose, an inert gas preferably selected from nitrogen, argon, CO₂, NO or a mixture thereof is particularly suitable as the gas for this gas stream.

If a gas stream is used to discharge the pyrolysate, it is preferable according to the invention when the flow rate of the gas stream in the reactor, as the superficial velocity, is in the range from 0.01 m/s to 20 m/s. If a fixed-bed reactor is chosen as the reactor, it is preferable according to the invention when the flow rate of the gas stream in the reactor, as the superficial velocity, is in the range from 0.03 m/s to 1 m/s. If a fluidized-bed reactor is chosen as the reactor, it is preferable according to the invention the flow rate of the gas stream in the reactor, as the superficial velocity, is in the range from 0.5 m/s to 2 m/s. If an entrained-flow reactor is chosen as the reactor, it is preferable according to the invention the flow rate of the gas stream in the reactor, as the superficial velocity, is in the range from 5 m/s to 20 m/s.

As the conditions of the invention for the decomposition in the pyrolysis in step (b), the temperature in the reactor is 200° C. to 350° C. and the amount of oxygen gas in the reactor is from 0% to 2.0% by volume based on the total volume of the gases present in the reactor.

The amount of oxygen gas according to the invention is established by filling the reactor packed with said material of said pyrolysis feedstock with inert gas, more particularly with nitrogen, argon, CO₂, NO or a mixture thereof. The inert gas may additionally be admixed with reactive gases other than oxygen gas, in particular gases selected from methane, gaseous H₂O, hydrogen gas or mixtures thereof.

To minimize the ingress of oxygen gas due to the introduction of the pyrolysis feedstock into the reactor, the pyrolysis feedstock can be freed from oxygen gas before it is introduced in step (a), for example by driving out the oxygen gas through stripping with a stripping gas, for example in a reservoir vessel upstream of the reactor. For example, an inert gas, more particularly nitrogen, argon, CO₂, NO or mixtures thereof, could be passed as a stripping gas from the top or the bottom of the reservoir vessel (preferably from the top) via a frit into the vessel and into the pyrolysis feedstock so as to drive out the oxygen gas.

If the polymeric compound is in the form of a foam, especially in the form of a flexible polyurethane foam, the foam will be greatly reduced in volume by pressing or hot pressing (200 bar) and can then be cut or shredded into small particles. The foam processed in this way can then be introduced into the pyrolysis reactor more easily.

In a further preferred embodiment of the process, the absolute pressure in step (b) is not more than 1.2 bar.

In a further preferred embodiment of the process, the absolute pressure in step (b) is at least 0.8 bar.

It has also been found to be generally even more advantageous when the process control is configured such that the temperature in step (b) is not set above 300°° C. Thus, in a further preferred embodiment of the process of the invention, the temperature in step (b) is between 200° C. and 300° C., further preferably from 230° C. to 295° C., more preferably from 250° C. to 290° C.

In a further preferred embodiment of the process of the invention, the amount of oxygen gas in the reactor in step (b) is not more than 0.5% by volume, preferably not more than 0.1% by volume, in each case based on the total volume of the gases present in the reactor.

In a very preferred embodiment of the process of the invention, firstly the temperature in step (b) is between 200° C. and 300° C., further preferably from 230° C. to 295° C., more preferably from 250° C. to 290° C., and secondly the amount of oxygen gas in the reactor is not more than 0.5% by volume, preferably not more than 0.1% by volume, in each case based on the total volume of the gases present in the reactor. It is in turn exceptionally preferable when, in addition, thirdly the absolute pressure in step (b) is from 0.8 to 1.2 bar.

A preferred embodiment of the process provides continuous process control. For this, at least steps (a) and (b) run concomitantly in the context of continuous process control.

The pyrolysis product obtained according to step (c) can be worked up using standard separation methods, for example distillation or selective condensation, thereby affording (i).

The discharged pyrolysis residue had been found to contain a large amount of compounds attributable to the further structural units present in the original polymeric compound of the pyrolysis feedstock, in particular the polyol structural units. In a sequential pyrolysis process that is according to the invention preferable, it has proven advantageous when the discharged pyrolysis residue is additionally subjected to a second pyrolytic decomposition at a temperature of more than 360° C. A preferred embodiment of the process of the invention is thus characterized in that in a further step the pyrolysis residue discharged from the reactor is introduced into a second reactor in which it is broken down at a temperature of more than 360° C. to obtain a second pyrolysate that is in the gas phase and a second pyrolysis residue that is not in the gas phase, wherein the amount of oxygen gas in the reactor during this decomposition is not more than 2.0% by volume based on the total volume of the gases present in the second reactor and wherein, during this decomposition, the second pyrolysate is discharged from the reactor and said second pyrolysis residue is discharged from the reactor.

It has been found to be preferable when the polymeric compound present in the pyrolysis feedstock of the preceding pyrolysis had been obtained by reaction at least of
i1) at least one organic isocyanate compound containing at least two to four, especially two, isocyanate groups attached to a hydrocarbon unit having 2 to 8 carbon atoms, preferably having 3 to 8 carbon atoms: with
i2) at least one organic compound having at least two hydroxy groups.

All of the aforementioned preferred embodiments of this reaction of il) with i2) are likewise considered to be preferably suitable in this embodiment.

In the aforementioned sequential embodiment of the process it is particularly preferable when the decomposition in the second reactor is at least 360° C., especially more than 400° C. Depending on the catalyst and the reactor regimen, it is possible for either propene and ethene to be obtained here, which can be converted into propylene oxide and ethylene oxide respectively or into an aromatic compound (preferably into an aromatic hydrocarbon compound having 6 to 10 carbon atoms, very particularly preferably into benzene, toluene, xylene or mixtures thereof).

The process of the invention can be carried out with the aid of a suitably configured pyrolysis device. The invention therefore further provides the pyrolysis device and for the use of a pyrolysis device comprising at least one metering device for feeding in pyrolysis feedstock, at least one heatable reactor for the pyrolysis, and at least one pyrolysate collector, wherein
said heatable reactor for the pyrolysis includes at least one heating element that can be used for temperature control of the reactor at a temperature of 200° C. to 350° C. and also at least one inlet for pyrolysis feedstock and at least one separate outlet for pyrolysate and at least one separate outlet for pyrolysis residue: and that
the metering device and heatable reactor for the pyrolysis are arranged and configured in relation to one another such that the metering device is connected via at least one supply line to an inlet for the pyrolysis feedstock of said reactor; and that
the heatable reactor for the pyrolysis and the pyrolysate collector are in fluid communication with one another such that it is possible for the preferably gaseous pyrolysate to be discharged from said outlet for pyrolysate and for the discharged pyrolysate to be introduced into the pyrolysate collector: and that
at least one pyrolysate collector includes at least one cooling device, temperature-controlled at a temperature below 200° C., that in said collector can be used to lower the temperature of the pyrolysate discharged from said reactor to less than 200° C., with the formation of a pyrolysis product selected from pyrolysate condensate, pyrolysate sublimate or a mixture thereof, and includes at least one container for collecting and discharging the pyrolysis product obtained by cooling: and that
at least one metering device for feeding in pyrolysis feedstock, at least one heatable reactor for the pyrolysis, and at least one pyrolysate collector are arranged and configured in relation to one another such that they can be operated concomitantly,
   for obtaining an organic compound having at least one amino group through pyrolysis of polymeric compounds having at least one polyurethane structural unit of the formula (I),

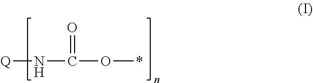

where
is a hydrocarbon radical having 2 to 8 carbon atoms, especially 3 to 8 carbon atoms, and
n is a number from 2 to 4, preferably 2,
and -* denotes a covalent bond to the polymer backbone.

The polymeric compounds mentioned in the description of the process are likewise considered to be preferred polymeric compounds for the pyrolysis.

It is in accordance with the invention preferable to use said pyrolysis device for the recovery of at least one aromatic amino compound having at least one amino group, selected in particular from aniline, toluidine, TDA or mixtures thereof.

The heating unit used for the heatable reactor of said pyrolysis device may for example be a heating element, for example a heating coil or heating plates, or a device for heating a gas stream and for introducing the heated gas stream into the reactor.

In a preferred embodiment, the reactor additionally includes at least one connection to a gas source with which a gas stream in the reactor, preferably having a flow rate, as the superficial velocity, of between 0.01 m/s and 20 m/s, flows via a regulator, for example a valve, through the reactor into the pyrolysate collector. If a fixed-bed reactor is chosen as the reactor, it is preferable according to the invention when the flow rate of the gas stream in the reactor, as the superficial velocity, is in the range from 0.03 m/s to 1 m/s. If a fluidized-bed reactor is chosen as the reactor, it is preferable according to the invention the flow rate of the gas stream in the reactor, as the superficial velocity, is in the range from 0.5 m/s to 2 m/s. If an entrained-flow reactor is chosen as the reactor, it is preferable according to the invention the flow rate of the gas stream in the reactor, as the superficial velocity, is in the range from 5 m/s to 20 m/s.

The gas stream from the gas source can for example be heated by the heating unit before it is introduced into the reactor as described above.

The pyrolysate collector of the pyrolysis device preferably includes a cooling device that in said collector can be used to lower the temperature of the pyrolysate discharged from said reactor to less than 50° C. (more preferably to less than 30° C.), with the formation of a pyrolysis product. Cooling units that work according to the heat exchanger principle are particularly suitable for this. The pyrolysate collector can here be fitted out as a selective condenser for a selective condensation of pyrolysis product constituents present in the pyrolysate.

A “fluid connection” is in accordance with the invention understood as meaning a part of the device that connects parts of the system to one another and through which a substance that may be in any state of matter can be transported from one plant component to the next, for example a supply line in the form of a pipe.

In addition, it is in a further embodiment of the pyrolysis device preferable when said pyrolysis device additionally contains
at least one further heatable reactor for the pyrolysis that includes at least one heating element that can be used for temperature control of the reactor at a temperature of above 360° C. and also at least one inlet for pyrolysis feedstock and at least one separate outlet for pyrolysate: and
with the proviso that the outlet for pyrolysis residue of said heatable reactor is in fluid communication with the inlet for pyrolysis feedstock of the further heatable reactor.

In this embodiment, it is preferable when the pyrolysis device includes at least one further pyrolysate collector that is in fluid communication with the further heatable reactor such that it is possible for the preferably gaseous pyrolysate to be discharged from said outlet for pyrolysate and for the discharged pyrolysate to be introduced into the pyrolysate collector.

In addition, said further pyrolysate collector is preferably designed such that it includes at least one cooling device, temperature-controlled at a temperature below 200° C., that in said collector can be used to lower the temperature of the pyrolysate discharged from said reactor to less than 200° C., with the formation of a pyrolysis product selected from pyrolysate condensate, pyrolysate sublimate or a mixture thereof, and includes at least one container for collecting and discharging the pyrolysis product obtained by cooling.

### EXAMPLES

### a) Providing the Pyrolysis Feedstock

A flexible polyurethane foam was produced by standard processes from the components shown in Table 1.

TABLE 1
Components of the flexible foam:

| | % by wt. |
|---|---|
| Constituents | |
| Polyol having an OH value of 48 | 64.26 |
| Water | 2.44 |
| Stabilizer | 0.64 |
| Dye | 0.32 |
| Catalyst mixture of tin octanoate, 1,4- | 0.16 |
| diazabicyclo[2.2.2]octane, and bis(2-(N,N- | |
| dimethylamino)ethyl) ether | |
| Isocyanate | |
| Desmodur ® T80 ¹ | 32.18 |

¹ Isocyanate from Covestro Deutschland AG, tolylene 2,4- and 2,6-diisocyanate (TDI) in a ratio of 80:20

To provide pyrolysis feedstock, the flexible foam was pressed with a heating press at 150° C. to 1/20 of its original height and then cut into 1 mm pieces. 2 g amounts of these pieces were then introduced into the reactor in five Al₂O₃ crucibles with holder. The pyrolysis was carried out as described in b).

### b) Pyrolysis Procedure

The pyrolysis of the flexible polyurethane foam was carried out at 273° C. in a fixed-bed reactor having a volume of 25 ml with a through-flow of Ar.

The flow rate of the nitrogen gas stream (superficial velocity) in the reactor was 0.07 m/s. The prepared pyrolysis feedstock was introduced into the reactor. The residence time of the introduced polyurethane material was 30 min. The reactor was heated to 273° C. and then held at this temperature for 30 min. Situated downstream of the reactor were two condensers for the separation of the liquid components of the resulting pyrolysis gas. The resulting content of carbonized material was determined by weighing the Al₂O₃ crucible after the pyrolysis. The gas downstream of the two condensers was characterized by online GC. The components of the pyrolysis product obtained in the form of an oil were determined by GC-FID. This was done using an Agilent 7890A with a Supelco SPB 50 column. The pyrolysis oil was diluted 1:50 or 1:100 with acetone. The results are illustrated in Table 2.

The pyrolysis residue obtained from this pyrolysis was pyrolyzed again. This second pyrolysis was carried out at 360° C. in a fixed-bed reactor having a volume of 25 ml with a through-flow of N₂.

The flow rate of the nitrogen gas stream (superficial velocity) in the reactor was 0.07 m/s. The pyrolysis residue from the previous pyrolysis was introduced into the reactor as pyrolysis feedstock. The residence time of the introduced pyrolysis feedstock was 30 min. The reactor was heated to 360° C. and then held at this temperature for 30 min. Situated downstream of the reactor were two condensers for the separation of the liquid components of the resulting pyrolysis gas.

TABLE 2
Composition of the pyrolysis product obtained, of the residual
gas, and of the pyrolysis residue from the pyrolysis of
the flexible polyurethane foam at 273° C. [% by wt.]:

| Pyrolysis | % by wt. |
|---|---|
| Pyrolysis product (oil): | |
| TDA | 14.62 |
| Residual oil (not characterized) | 11.90 |
| Gas phase after condensation | |
| CO₂ | 4.00 |
| CO | 0.04 |
| Propene | 3.27 |
| Other gases | 2.51 |
| Pyrolysis residue | |
| Polyol with carbonized material | 63.66 |
| Total | 100.00 |

1. A pyrolysis process, comprising:
(a) introducing into a reactor a pyrolysis feedstock comprising at least one polymeric compound having at least one polyurethane structural unit of the formula (I), 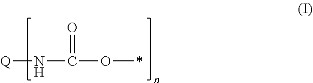
where
Q is a hydrocarbon radical having 2 to 8 carbon atoms,
n is a number from 2 to 4, and
-* denotes a covalent bond to the polymer backbone;
(b) decomposing at least the polymeric compound of the pyrolysis feedstock introduced in step (a) in the reactor at a temperature of 200° C. to 350° C. to obtain a gas-phase product as pyrolysate and a non-gas-phase pyrolysis residue, wherein
(i) during said decomposition, the amount of oxygen gas in the reactor is not more than 2.0% by volume based on the total volume of the gases present in the reactor,
(ii) during said decomposition, the pyrolysate is discharged from the reactor, and
(iii) said pyrolysis residue is discharged from the reactor;
(c) cooling the discharged pyrolysate to a temperature of less than 200° C. to obtain pyrolysis product selected from pyrolysate condensate, pyrolysate sublimate or a mixture thereof; and
(d) optionally working up the pyrolysis product.

2. The process as claimed in claim 1, wherein the introduced pyrolysis feedstock is temperature-controlled at a target temperature of 200° C. to 350° C. and, on reaching the target temperature, the residence time of the temperature-controlled pyrolysis feedstock until the time of discharge of the pyrolysis residue is from 1 second to 5 hours.

3. The process as claimed in claim 1, wherein in that the temperature in step (b) is between 200° C. and 300° C.

4. The process as claimed claim 1, wherein the decomposition takes place at an absolute pressure of not more than 1.2 bar.

5. The process as claimed in claim 4, wherein the absolute pressure is at least 0.8 bar.

6. The process as claimed in claim 1, wherein the discharge of the pyrolysate from the reactor achieved by passing a gas stream through the reactor or by suction.

7. The process as claimed in claim 6, wherein the discharge of the pyrolysate from the reactor is achieved by passing a gas stream through the reactor, in which the flow rate of the gas stream in the reactor, as the superficial velocity, is in the range from 0.01 m/s to 20 m/s.

8. The process as claimed in claim 1, wherein at least steps (a) and (b) run concomitantly in the context of continuous process control.

9. The process as claimed in claim 1, wherein the amount of oxygen gas in the reactor in step (b) is not more than 0.5% by volume, based on the total volume of the gases present in the reactor.

10. The process as claimed in claim 1, wherein an inert gas is passed through the reactor packed with said pyrolysis feedstock.

11. The process as claimed in claim 1, wherein the polymeric compound contains at least one polyurethane structural unit of the formula (Ia) 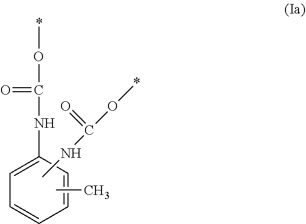
where
-* is a covalent bond to the polymer backbone.

12. The process as claimed in claim 1, wherein the polymeric compound is a reaction product of at least
i1) at least one organic isocyanate compound containing two to four, isocyanate groups attached to a hydrocarbon unit having 2 to 8 carbon atoms; with
i2) at least one organic compound having at least two hydroxy groups.

13. The process as claimed in claim 12, wherein the at least one organic isocyanate compound contains, as said hydrocarbon unit, a unit that has 2 to 8 carbon atoms and is derived from aliphatic hydrocarbon units, cycloaliphatic hydrocarbon units, araliphatic hydrocarbon units, aromatic hydrocarbon units or heterocyclic hydrocarbon units.

14. The process as claimed in claim 12, wherein the at least one organic isocyanate compound comprises at least one diisocyanate of the formula (III),

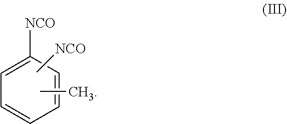

15. The process as claimed in claim 1, wherein said polymeric compound is introduced into the reactor as a constituent of a material in the form of solid particles.

16. The process as claimed in claim 1, wherein in a further step the pyrolysis residue discharged from the reactor is introduced into a second reactor in which it is decomposed at a temperature of more than 360° C. to obtain a second pyrolysate that is in the gas phase and a second pyrolysis residue that is not in the gas phase, wherein the amount of oxygen gas in the second reactor during the decomposition in the second reactor is not more than 2.0% by volume based on the total volume of the gases present in the second reactor and wherein, during the decomposition in the second reactor, the second pyrolysate is discharged from the second reactor and said second pyrolysis residue is discharged from the second reactor.

17. The process as claimed in claim 16, wherein the second pyrolysate is subjected to a workup to provide a resulting product gas that is supplied to a third reactor in which the resulting product gas is converted into alkylene oxide.

18. A process for obtaining an organic compound having at least one amino group from a pyrolysis feedstock, the method comprising:
feeding the pyrolysis feedstock to a pyrolysis device comprising at least one metering device for feeding in pyrolysis feedstock, at least one heatable reactor for the pyrolysis, and at least one pyrolysate collector, wherein
said heatable reactor for the pyrolysis includes at least one heating element that is used for temperature control of the reactor at a temperature of 200° C. to 350° C., at least one inlet for pyrolysis feedstock, and at least one separate outlet for pyrolysate;
the metering device and heatable reactor for the pyrolysis are arranged and configured in relation to one another such that the metering device is connected via at least one supply line to an inlet for the pyrolysis feedstock of said reactor;
the heatable reactor for the pyrolysis and the pyrolysate collector are in fluid communication with one another such that a pyrolysate discharged from said outlet for pyrolysate and for the discharged pyrolysate are introduced into the pyrolysate collector;
at least one pyrolysate collector includes at least one cooling device, temperature-controlled at a temperature below 200° C., that in said collector lowers the temperature of the pyrolysate discharged from said reactor to less than 200° C., with the formation of a pyrolysis product selected from pyrolysate condensate, pyrolysate sublimate or a mixture thereof, and includes at least one container for collecting and discharging the pyrolysis product obtained by cooling; and
at least one metering device for feeding in pyrolysis feedstock, at least one heatable reactor for the pyrolysis, and at least one pyrolysate collector are arranged and configured in relation to one another such that they are operated concomitantly, and
pyrolysis feedstock comprises polymeric compounds having at least one polyurethane structural unit of the formula (I), 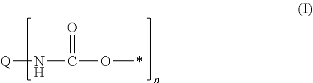
where
Q is a hydrocarbon radical having 3 to 8 carbon atoms
n is a number from 2 to 4, and
-* denotes a covalent bond to the polymer backbone.
